(19) United States
(12) Patent Application Publication
(10) Pub. No.: US 2007/0269883 A1
(43) Pub. Date: Nov. 22, 2007

(54) Micropatterning surfaces

(76) Inventor: Kathryn Uhrich, Plainfield, NJ (US)

Correspondence Address: VIKSNINS HARRIS & PADYS PLLP, P.O. BOX 111098, ST. PAUL, MN, 55111-1098, US

(21) Appl. No.: 11/435,812

(22) Filed: May 16, 2006

(51) Int. Cl. C12M 3/00

(52) U.S. Cl. 435/287.2; 427/2.11; 977/902

(57) ABSTRACT

The present invention is directed to a method for preparing a surface having a pre-selected ink pattern.

### PRIORITY OF INVENTION

This application claims priority under 35 U.S.C. §119(e) to U.S. Provisional Application No. titled “Micropatterning Surfaces using Hydrophyllic/Hydrophobic Segregated Areas Produced by Selective Plasma Treatment,” which was filed 16 May 2005 in the name of Kathryn Uhrich. The entire content of this provisional application is incorporated herein by reference.

### FIELD OF THE INVENTION

The present invention relates generally to the field of making patterns on substrates. More specifically, it relates to a method for making an article with a pattern (e.g. a pattern of biologically active molecules) on the surface of a substrate (e.g. a polymeric substrate) and to articles made using the method.

### BACKGROUND OF THE INVENTION

Micropatterning is a technique that has been in use for decades in the computer industry for patterning of microchips. More recently, patterning of substrates has been contemplated for biological applications including biological assays, medical implants and articles for adhering and growing cells. Most of such micropatterning techniques have involved placing patterns on inorganic, non-polymeric substrates such as glass.

High-resolution photolithography, sometimes referred to as microlithography, and focused laser methods have been described for patterning surfaces with molecular layers (Dontha et al., Anal. Chem., 69:2619 (1997)); Kleinfeld et al., J. Neurosci., 8:4098 (1988)). However, photolithography requires the use of harsh solvents and bases which are incompatible with many biological molecules, and the laser method uses an interference technique that does not permit generation of patterns of arbitrary complexity (James et al., Langmuir, 14:741 (1998)).

Microcontact printing is another relatively new technique that has been used to produce micron-sized features on inorganic surfaces. Using this technique, inorganic substrates, such as glass and silicon oxide, have been micropatterned by stamping inks such as alkanethiols on their surface (St. John et al., Anal. Chem., 70:1108 (1998); and James et al., Langmuir, 12:741 (1998)). In this method, a high resolution protein pattern is applied to the surface using a stamp made from poly(dimethylsiloxane) secured to a glass backing material.

One instance has been described where a biocompatible polymer having a micro-patterned surface was produced (WO 99/36107). In this case, a ligand was attached to a biocompatible polymer though a specific biotin-avidin linkage.

In spite of the above reports, there remains a need for improved (e.g. faster, simpler, less expensive) methods for preparing articles having a pattern on their surface (e.g. articles having a pattern of biologically active molecules on a substrate).

### SUMMARY OF THE INVENTION

The present invention provides a method comprising: providing a surface including a plurality of areas having different surface energies, and contacting the surface with an ink that preferentially adheres to one area.

The invention also provides a method comprising: providing a surface that has a protected portion and an unprotected portion; and treating the surface under conditions that alter the surface energy of the unprotected portion to provide a surface including a plurality of areas having different surface energies.

The present invention also provides a method comprising: providing a surface that has a protected portion and an unprotected portion; treating the surface under conditions that alter the surface energy of the unprotected portion to provide a surface including two areas having different surface energies, and contacting the surface with an ink that preferentially adheres to one of the two areas.

The present invention also provides a method comprising: contacting a stamp having a pre-selected pattern with a surface to provide a surface that has a portion that is protected by the stamp and an unprotected portion; treating the surface under conditions that alter the surface energy of the unprotected portion to provide a surface including two areas having different surface energies, and contacting the surface with an ink that preferentially adheres to one of the two areas. The stamp can optionally be removed from contact with the surface either prior to contact with the ink or after contact with the ink. Additionally, non-adhered ink can optionally be washed from surface following contact with the ink.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1. Illustrates patterning method of the invention that can be used to generate simple biomolecular micropatterns.

FIG. 2. Illustrates patterning method of the invention that can be used to generate complex biomolecular micropatterns.

FIG. 3. Illustrates fluorescent micrographs of simple patterns generated by the method illustrated in FIG. 1: (a) BSA on PDMS, (b) BSA on PHBV, (c) poly-L-lysine on PMMA, (d) poly-L-lysine on PE, and (e) α-rabbit IgG on PS. The scale bar indicates 50 μm.

FIG. 4. Fluorescent micrograph that shows the extent of pattern formation by the method illustrated in FIG. 1 for BSA on PS. The scale bar indicates 250 μm.

FIG. 5. Fluorescent micrographs of complex patterns generated by the method outlined in FIG. 2: (a) poly-L-lysine on PMMA, (b) α-rabbit IgG on PDMS, (c) BSA on PS, and (d) poly-L-lysine on PDMS. The scale bar indicates 50 μm.

FIG. 6. Fluorescent micrographs of BSA patterns on PS (a & b) and PDMS (c & d). Images a and c were taken immediately after patterning, whereas images b and d were taken following four weeks of storage in PBS at 37° C. Images a and b are from the same area on the same sample, whereas images c and d are from different areas on the same sample. The scale bar indicates 100 μm.

FIG. 7. Fluorescent micrograph of PMMA patterned with poly-L-lysine (green) and BSA (red) by immersion in a mixture of both inks. Scale bar indicates 50 μm.

FIG. 8. SEM micrographs of various substrates following exposure to oxygen plasma while in contact with a patterned PDMS stamp: (a) PMMA (620×), (b) PDMS (655×), and (c) PHBV (1180×). Encircled regions denote areas that were protected from plasma. SEM micrograph (d) of the patterned PDMS stamp (1180×) in contact with PHBV (c) during oxygen plasma treatment.

### DETAILED DESCRIPTION OF THE INVENTION

The methods of the invention are useful for preparing articles having a pattern of ink on a surface. Such articles are useful for printing and circuit design, as well as for biological and medical applications.

Micropatterned biomolecules have a number of applications, for example, modulating cell-substrate interactions, spatially directing cell growth, tissue regeneration, combinatorial screening strategies, and multiple analytical biosensors. Some of the most exciting applications of patterned biologically active molecules on biocompatible substrates include modulating the growth of cells through cell-substrate interactions. Using the methods of the invention, cell growth can be modulated spatially both in vivo and in vitro, so that for example, an injured tissue can be regenerated into an appropriate, functional shape, and a severed nerve cell connection can be repaired by encouraging nerve cells to grow across a gap in the connection. Also, the rate of cell growth can be modulated through use of biologically active molecules that increase cell growth, e.g. growth factors.

Preferred embodiments of the present invention include a method comprising:

a) contacting a stamp having a pre-selected pattern with a surface to provide a surface that has a portion that is protected by the stamp and an unprotected portion;

b) treating the surface under conditions that alter the surface energy of the unprotected portion to provide a surface including two areas having different surface energies, and

c) contacting the surface with an ink that preferentially adheres to one of the two areas; and

d) optionally removing the stamp from contact with the surface either prior to contact with the ink or after contact with the ink; and

e) washing non-adhered ink from surface following contact with the ink.

An article made from a substrate with a stably adsorbed pattern of biologically active molecules exhibits many advantages. One advantage of adsorption is that linker molecules between the substrate and the biologically active molecules are not needed. A second advantage is that the article has fewer components and will generate fewer immunogenic responses and fewer side effects. A third advantage is that manufacturing procedure is simple, fast, inexpensive and avoids coupling agents and other harsh chemicals which may create negative side effects when left in the article in even small amounts. There is no need for UV exposure, high processing temperatures, organic solvents, expensive equipment, extensive manufacturing space and/or lengthy production times. Finally, the present methods produce a substrate surface which comprises distinct regions of surface energies that are preferably, but not limited to, hydrophobic/hydrophilic regions. Articles with patterns of biologically active molecules that are surrounded by regions of hydrophobicity tend to encourage specific interaction of biomolecules and cells with the stably adsorbed biologically active molecules. Nonspecific binding of biomolecules and cells to the unbound surface of the substrate is discouraged because that surface is hydrophobic.

Definitions

The term “ink” includes atoms and molecules that will selectively adhere to a hydrophobic surface or to a hydrophilic surface. For example, the term includes colored materials, radioactive materials, magnetic materials, conductive materials, insulating materials, and biologically active molecules.

The term “biologically active molecule” is used herein to denote a molecule that can be stably adsorbed on the surface of the substrate and which has a useful in vivo or in vitro function. In general, such biologically active molecules can have an effect on a biological process such as cell adhesion, cell growth, cell-to-cell contact or communication and the like. Typically, portions of the biologically active molecules will be polar or somewhat hydrophilic in nature so that they can adsorb into the plasma-activated substrate. Hence, biologically active molecules having functional groups which have a dipole moment, such as amines, amides, carbonyls, carboxylates, esters, alcohols, sulfhydryls and the like are particularly suited for use in the present invention. Examples of biologically active molecules for use in the present invention include hormones, extracellular matrix molecules, cell adhesion molecules, natural polymers, enzymes, peptides, antibodies, antigens, polynucleotides, growth factors, synthetic polymers, polylysine, drugs and other molecules. Additional specific examples are provided below.

As used herein, the term “hydrophilic” refers to a general affinity for a chemical group to attract water or to otherwise exhibit sufficient polarity to permit stable adsorption to another polar group. The term can be used to describe chemical groups in both the substrate and the biologically active molecules and includes groups such as amines, amides, carbonyls, carboxylates, esters, alcohols, sulfhydryls and the like.

The term “micron-sized,” as used herein, means that a pattern or article is microscopic in size. In general, micron-sized patterns and articles are about 10 nm to about 1000 microns in size. Preferably micron-sized patterns are about 0.1 microns to about 500 microns in size. More preferably, micron-sized patterns are about 1 micron to about 100 microns in size. Most preferred patterns and articles are about 1 micron to about 50 microns in size.

As used herein, a “plasma” is an ionized gas capable of transiently modifying a substrate. Any such plasma known to one of skill in the art can be used in the present invention. Examples of plasmas which can be used include argon, nitrogen, oxygen, and other plasmas.

The term “polar,” as used herein, refers to groups on the surface of the substrate and on the biologically active molecules of the invention which have a dipole moment. For example, the substrate may be made polar by modifying the surface energy within the substrate or by temporarily aligning the dipole moments or polarity of the molecules within the substrate. The term can be used to describe chemical groups in the substrate and in the inks and includes groups such as amines, am ides, carbonyls, carboxylates, esters, alcohols, sulfhydryls and the like.

As provided herein, the term “substrate” includes materials that have sufficient mechanical stability and strength to serve as the structural material of an article of the present invention, as well as coatings (e.g. polymer coatings) on such materials, which are capable of developing a transiently modified surface energies when treated with a plasma according to the methods of the present invention. The term includes includes glasses and silicone, as well as polymeric substrates.

The term “polymeric substrate” includes polymers which have sufficient mechanical stability and strength to serve as the structural material of an article of the present invention and which are capable of becoming transiently modified (e.g. activated) when treated with a plasma according to the methods of the present invention. Generally, the present polymeric substrates are hydrophobic before treatment with a plasma and become sufficiently activated to adsorb biologically active molecules when treated with a plasma. However, after such treatment the surface of the present polymeric substrate will return to its original state, and become more hydrophobic unless that surface has adsorbed biologically active molecules. Polymers selected for use in the present articles are preferably biocompatible and are preferably not polydimethylsiloxane. Examples of polymers for use in the present invention are provided below.

As used herein, a “pre-selected pattern” can be any pattern, such as, for example, lines, circles, ovals, squares, rectangles, diamonds, triangles or a combination of any of these shapes. A “pre-selected pattern” for a biological molecule can be a shape useful for adhesion, detection, growth or identification of other molecular species, macromolecules, biomolecules, cells and tissues. Hence, while a mixture of biologically active molecules will adopt a “pre-selected pattern,” other molecular species, macromolecules, biomolecules, cells and tissues which bind to or interact with those biologically active molecules need not initially adopt that pattern. Instead, such molecular species, macromolecules, biomolecules, cells and tissues are only encouraged to grow into or otherwise adopt such a pre-selected pattern, for example, as time progresses.

As provided herein, the term “stably adsorbed” means that the ink is bound to the surface of the substrate by nonspecific molecular interaction. Inks which are stably adsorbed to the substrate will remain adsorbed for a sufficient time and with a sufficient affinity to be used for a selected application.

As used herein, a “stamp” is an object having a pre-selected pattern. Typically, the pre-selected pattern is physically raised from a surface of the stamp. When used according to the methods of the invention, the pre-selected pattern on the stamp makes contact with a surface of the substrate and protects a corresponding pattern on the surface of the substrate from the plasma.

### An Article Having a Pattern of Biologically Active Molecules.

The present invention allows for the preparation of an article having a pre-selected pattern of biologically active molecules stably adsorbed directly onto a substrate. The pattern typically includes a bound substrate surface having biologically active molecules stably adsorbed thereto and an unbound substrate surface having substantially no biologically active molecules adsorbed thereto. While any size article and pattern is contemplated, micron-sized articles and patterns are particularly useful for many biological and medical applications. The final articles typically have a pattern of biologically active molecules stably adsorbed to their surfaces which can be used for a variety of purposes including biological testing and assays, tissue regeneration, micropatterning and directing the spatial alignment of biomolecules and cells which adhere to the biologically active molecules.

The biologically active molecules are stably adsorbed directly onto a substrate by nonspecific molecular interaction. Such stable adsorption by nonspecific molecular interaction is accomplished by activating the surface of the substrate so that the biologically active molecules can be adsorbed to it.

The surface of the substrate can have any shape, for example, it can be flat, curved or tubular. In general, the surface of the substrate is not raised; instead, it is preferably a flat or curved planar surface. For purposes of this invention, the substrate can be biodegradable or nondegradable. Typically, to be useful in both in vivo and in vitro applications, the substrates are nontoxic, biocompatible, processable, transparent for microscopic analysis and mechanically stable.

### Polymers for Use with the Invention.

The main criteria used to select a polymer substrate is that the polymer is capable of becoming activated toward adsorption of the biologically active molecules of the present invention (e.g., the surface of the polymer is transiently activated) when treated with a plasma. The substrates are generally hydrophobic before treatment with the plasma and, unless adsorbed to biologically active molecules, will return to being hydrophobic after the adsorption process is completed. In one embodiment of the invention, the polymeric substrate is not polydimethylsiloxane.

A large variety of polymers may be used as substrates. One of skill in the art can readily select an appropriate polymeric substrate with sufficient mechanical stability and properties to be used in the methods of the present invention. Examples of polymers useful for the present articles and methods include polyacrylates, polymethylacrylates, polycarbonates, polystyrenes, polysulphones, polyhydroxy acids, polyanhydrides, polyorthoesters, polyphosphazenes, polyphosphates, polyesters, nylons or mixtures thereof. Examples of polymers of poly(hydroxy acids) include poly(hydroxybutyric acid), poly(lactic acid), poly(glycolic acid) and poly(caproic acid). Polyanhydrides, polyorthoesters, polyphosphazenes, polyphosphates, polycaprolactone or copolymers prepared from the monomers of these polymers (see for example WO 95/03357) may also be used. Poly(ortho-esters), polyol/diketene acetals and related polymers are provided by Heller, ACS Symposium Series 567, 292-305, 1994. Examples of biodegradable hydrophobic polyanhydrides are disclosed, for example, in U.S. Pat. No. 4,757,128; U.S. Pat. No. 4,857,311; U.S. Pat. No. 4,888,176 and U.S. Pat. No. 4,789,724. Polyhydroxybutyrates are disclosed in U.S. Pat. No. 3,044,942.

Polymers of lactic acid or glycolic acid, or copolymers of these monomers are contemplated, such as poly(lactic acid), poly(glycolic acid) or poly(lactic-co glycolic) acid., poly(E-caprolactone), poly(3-hydroxybutyrate), poly(p-dioxanone), polyalkylene glycol, polyethylene and polypropylene fumarate.

Polyanhydrides for use as the polymeric substrate of the present invention include, but are not limited to: poly(sebacic anhydride), poly(carboxybiscarboxyphenoxyphenoxyhexane), poly[bis(p-carboxyphenoxy)methane], and copolymers thereof which are described by Tamada and Langer in Journal of BioMaterials Science Polymer Edition, 3:315 (1992) and by Domb in Chapter 8 of the Handbook of Biodegradable Polymers, ed. Domb A. J. and Wiseman R. M., Harwood Academic Publishers. Also contemplated are poly(amino acids), and poly(pseudo amino acids) that include those described by James and Kohn in pages 389-403 of Controlled Drug Delivery Challenges and Strategies, American Chemical Society, Washington D.C. Polyphosphazenes for use in the present invention include derivatives of poly[(dichloro)phosphazene]poly[(organo)phosphazenes]polymers described by Schacht in Biotechnology and Bioengineering, 52, 102-108, 1996.

In a preferred embodiment, polyesters of poly(lactic-co-glycolic)acid (“PLGA”) are used. These polymers are approved for parenteral administration by the FDA. Because PLGA degrades via non-enzymatic hydrolysis in the initial stages, in vivo degradation rates can be predicted from in vitro data. PLGA is also a desirable substrate because it degrades to lactic and glycolic acids, substances found naturally in the body.

Additionally, copolymers with amino acids are contemplated as polymeric substrates of the present invention, for example, glycolic acid and glycine, or lactic acid and lysine as described in Barrera et al., J. Am. Chem. Soc., 115:11010 (1993) and Cook et al., J. Biomed. Mat. Res., 35:513 (1997). Biodegradable materials also include collagen and polysaccharide gels, for example, of hyaluronic acid. Copolymers of collagen and proteoglycans may also be used.

Protein polymers may also be used for the polymeric substrate and are prepared by available protein chemistry and molecular biology techniques. For example, polymers based on silk or elastin repeating units may be prepared and are suitable for use in the present invention (Hubbell J A., Biotechnology, 13:565 (1995)). It will be appreciated that some biocompatible polymers, for example, some natural polymers as described above, may degrade in response to cellular and enzymatic activity and that the rate of such degradation may vary depending on the environment or cultural conditions involved. While such degradation can be a useful property, particularly when the polymeric substrate is used in vivo, the rate of degradation by the polymeric substrate will preferably not be faster than the rate of tissue regeneration or the time needed for analytical testing. The rate of degradation in a specific environment can be observed by methods known to one of skill in the art. For example, the rate of degradation can be observed by placing the polymeric substrate in the environment in which it will be used and observed how long it remains intact. Hence, degradation can readily be observed and manipulated by one of skill in the art. In addition, natural polymers, such as collagen can be patterned using a method of the invention.

### Biologically Active Molecules

Biologically active molecule therefore include any molecule that can effect a biological process, such as cellular adhesion, growth or differentiation. Biologically active molecules that inhibit or promote growth and/or differentiation of a particular type of cell are contemplated. It is preferred that the biologically active molecule is a peptide, protein, carbohydrate, nucleic acid, lipid, polysaccharide, or combinations thereof, for example a proteoglycan, or synthetic inorganic or organic molecule. Examples of biologically active molecules for use in the present invention include hormones, extracellular matrix molecules, cell adhesion molecules, natural polymers, enzymes, peptides, antibodies, antigens, polynucleotides, growth factors, synthetic polymers, polylysine, drugs and other molecules.

The following biologically active molecules are understood to be exemplary and are not to be limiting in any manner. Examples of biologically active molecules that may be used include cell binding domains of extracellular matrix proteins and other adhesion proteins, for example fibronectin and vitronectin, or fragments thereof, that are recognized by cytoskeleton associated receptors in the cell membrane, known as integrins. Such receptors can bind to a small domain on the adhesion proteins, for example, the peptide sequence Arg-Gly-Asp (also referred to a “RGD”), which is found in many adhesion proteins, and which binds to many integrins. Varying the sequence or flanking sequences can alter the binding affinity of a receptor for the peptide or protein containing it. The density of the biologically active molecule in the pre-selected pattern may affect adhesion, binding and cellular responses, and it will be appreciated that it may be necessary to control the density of the biologically active molecule to get the optimum density for practicing the present methods.

Further examples pf biologically active molecules contemplated by the present invention include the peptide Tyr-Ile-Gly-Ser-Arg (SEQ ID NO:1), found in the B1 chain of laminin which binds to the 67 kDa laminin receptor found on many cell types, and the peptide Ile-Lys-Val-Ala-Val (SEQ ID NO:2) found in the A chain of laminin which binds a 110 kDa receptor and which can induce neurite growth. Many different peptides with SEQ ID NO:2 sequence may stimulate neurite extension and any peptide that includes a sequence of amino acids that is able to bind to a cell adhesion receptor is contemplated by the present invention. For example, the isolated SEQ ID NO:2 peptide may not be sufficiently water soluble for all of the present applications. As an alternative, the water soluble peptide Cys-Ser-Arg-Ala-Arg-Lys-Gln-Ala-Ala-Ser-Ile-Lys-Val-Ala-Val-Ser-Ala-Asp-Arg (SEQ ID NO:3) may be used. The peptide Arg-Glu-Asp-Val (SEQ ID NO:4) from fibronectin binds to the integrin on human endothelial cells, but does not support adhesion or spreading of smooth muscle cells, fibroblasts or platelets and may therefore be useful for achieving selective cell adhesion.

Cell binding domain sequences of extracellular matrix proteins may also be used as biologically active molecules within the present invention. Examples of such domain sequences include: the Arg-Gly-Asp-Ser (SEQ ID NO:5) peptide sequence found in fibronectin which can mediate adhesion of most cells via the ap receptor; the Leu-Asp-Val and Arg-Glu-Asp-Val (SEQ ID NO:6) peptide sequences from fibronectin which can also mediate adhesion of cells; the Arg-Gly-Asp-Val (SEQ ID NO:7) peptide sequence from vitronectin which can mediate adhesion of most cell types via the ccp receptor; the Leu-Arg-Gly-Asp-Asn (SEQ ID NO:8) peptide sequence from Laminin A which can mediate cell adhesion; the Pro-Asp-Ser-Gly-Arg (SEQ ID NO:9) peptide from Laminin B1 which can mediate cell adhesion; the Arg-Asn-Ile-Ala-Glu-Ile-Ile-Lys (SEQ ID NO:13) peptide from Laminin B2 which can mediate neurite extension; the short Asp-Ala dipeptide sequence; the Arg-Gly-Asp-Thr (SEQ ID NO:10) peptide from Collagen I which can mediate adhesion of most cells; the Asp-Gly-Glu-Ala (SEQ ID NO:11) sequence which can mediate adhesion of platelets and other cells; and the Val-Thr-Xaa-Gly (SEQ ID NO:12) of thrombospondin which can mediate adhesion of platelets.

Further examples of biologically active molecules useful in the present invention include epidermal growth factor, nerve growth factor, insulin-like growth factor, basic fibroblast growth factor, platelet derived growth factor, transforming growth factor and related growth factors. Other examples include bone morphogenetic proteins, cytokines including interferons, interleukins, and monocyte chemotactic protein-1. It will be appreciated that the biologically active molecules of the present invention may also be provided on biocompatible, biodegradable polymeric substrates and so that they may be released as the material degrades.

Further examples of biologically active molecules contemplated by the present invention include dopamine, amine-rich oligopeptides, such as heparin binding domains found in adhesion proteins such as fibronectin and laminin. Other examples include amines, basic amino acids, and monosaccharides which can bind to the asialoglycoprotein receptor on hepatocytes. For example, one can stably adsorb N-acetylglucosamine or lactose or a polymerized N-acetyllactosamine monomer to the polymeric substrates of the present invention. Another example is sialyl Lewis X saccharide (Varki, Proc. Natl. Acad. Sci., (USA) 91:7390 (1994)) which is a biologically active molecule for the selectin class of saccharide-binding receptors that are usually responsible for mediating cell-cell interactions (Lasky, Science, 258:964 (1992)). Thus this saccharide may be useful for mimicking cell-cell recognition.

Bone morphogenetic proteins may be useful as biologically active molecules of the present invention for closure of defects in bone. Basic fibroblast growth factor is a useful biologically active molecule for inducing vascularisation. A slow release formulation, wherein the biologically active molecules are slowly released from the degrading polymer may be effective for these molecules.

The suitability of a biologically active molecule for use in the present invention may be assessed by methods known to those skilled in the art. For example, when it is desired to bind a specific biomolecule or cell to an article of the present invention, a potential biologically active molecule is stably adsorbed to a substrate and the binding or adhesion of a biomolecule or cell can be assessed by observing whether the cell or biomolecule binds to a substrate, by measuring protein-protein interactions between the biomolecule or cell and the biologically active molecules, or by detecting whether an antibody reactive with the biomolecule or cell becomes bound to the substrate. Functional assays for detecting whether a cell responds to a biologically active molecule may also be used, for example, functional assays capable of assessing whether the cell grows or adheres to the biologically active molecule under consideration. All of these procedures are available and can be adapted by one of skill in the art to identify biologically active molecules which are suitable for use in the present articles and methods.

### Methods for Preparing Surfaces Having Patterned Areas with Differing Surface Energies.

A substrate having a pre-selected pattern of areas with differing surface energies can be prepared using any suitable method. In one embodiment of the invention, such a substrate is prepared by protecting a pattern on the substrate while simultaneously exposing the unprotected parts of the substrate to an agent (e.g. a plasma) that modifies the affinity of the unprotected parts for the ink. The pattern on the substrate can be protected by physically blocking contact with the agent.

Any method known to one of skill in the art can be used to modify the affinity of the surface of the substrate for the ink. For example, the agent that modifies the affinity of the unprotected parts of the substrate for the ink can be a chemical agent, a plasma, or radiation.

In one specific embodiment of the invention, the substrate may be made polar by modifying the surface energy or surface tension of organic groups within the substrate or by temporarily aligning the dipole moments or polarity of the molecules within the substrate.

In one specific embodiment of the invention, the substrate is placed in a low temperature plasma generator and exposed to a plasma for a time and at a temperature and pressure which temporarily modifies the affinity of the substrate for the ink. For example, the substrate can be exposed to a stream of plasma for a temperature and under an electrical wattage sufficient to alter the surface energy of the exposed surface. The rate of exposure to plasma can vary depending on the type of plasma, the temperature and other factors. For example, a rate of about 0.01 to 100 cc/minute, preferably about 0.1 to 50 cc/minute and more preferably about 1-10 cc/minute can be used One of skill in the art can also vary the time of such exposure as needed to modify the surface energy of the substrate. For example, the time of exposure to plasma can include any suitable time, such as for example from about 0.5 to 300 seconds, preferably from about 1 to 200 seconds and more preferably from about 5-120 seconds. One of skill in the art can also readily determine a temperature sufficient to modify the surface energy of the substrate. For example, convenient temperatures are about 5° C. to about 42° C., preferably about 10° C. to about 37° C. and more preferably about room temperature. An electrical wattage sufficient to modify the surface energy of the surface of the substrate varies with the type of plasma. For example, such a wattage can vary from about 5 to 500 Watts, preferably about 50-400 Watts and more preferably about 100-300 Watts. Convenient conditions include exposing the article to oxygen plasma at a rate of about 4 cc/minute, for about 30 seconds at 200 Watts and at room temperature.

Any plasma known by one of skill in the art to activate the surface of the substrate can be used. Types of plasmas contemplated by the present invention include argon, nitrogen, oxygen, and other gases known to those of skill in the art to readily be ionized. Preferably the surface is treated with oxygen plasma.

Substrates treated in the manner describe can be evaluated via x-ray photoelectron spectroscopy, atomic force microscopy, and near-field scanning optical microscopy. The present methods typically do not alter the chemical composition of the substrate in a significant manner. However, the surface energy is transiently modified for a sufficient time to apply the ink, for example, for about 5 to about 60 minutes, after which time the unprotected substrate will typically return to its original state.

Transferring the ink to the surface of the substrate generally involves contacting that surface with the ink using any suitable method. For example, the ink can be coated, sprayed, or brushed onto the surface, or the surface can be dipped into a solution of the ink.

Upon pressing a stamp to the surface of the substrate, the pre-selected pattern is protected on the surface of the substrate. Using such a method, micron-sized patterns can be prepared on the substrate. The patterns can include lines having any widths. For example, for micron-sized patterns, the line can be about 0.01 to 10 microns in width, and about 10 to 500 microns in length. Preferably, such lines are about 0.1 to 1.0 microns in width and about 1 to 100 microns in length. More preferred line patterns are about 5 micron to about 50 microns in size.

A stamp can be made by any method known to one of skill in the art. One procedure for making a stamp involves the preparation of a master that has the reverse image of the pattern to be placed on the stamp. Briefly, a master may be fabricated on a polished silicon wafer using AS P4620 photoresist (Clariant, Inc.) which is spin coated to a thickness of about 5 mm and processed by contact photolithography. Methods for the production of masters are known in the art. See, Moread, W. M., Semiconductor Lithography: Principles and Materials, Plenum, N.Y. (1988); Brambley et al., Adv. Mater. Opt. Electron., 4:55 (1994); Handbook of Microlithography, Micromachining, and Microfabrication, Vol. 1 (Ed: P. Rai-Choudbury), SPEI Optical Engineering Press, Bellingham, Wash. (1997).

Any material known to one of skill in the art can be used to make the stamp. A preferred material for the stamp is poly(dimethyl siloxane) (PDMS). One source of PDMS material is Sylgard 184™ (Dow Corning, Midland, Mich.). The flexibility of a stamp prepared from this material permits patterning of non-flat surfaces such as round or cylindrical substrates. For PDMS stamp preparation, the PDMS material can be first prepared in liquid form and mixed with a curing agent. Bubbles are removed from the mixture, preferably via a vacuum at 28″ Hg, and the mixture is poured over a master pattern. It is preferred that the master pattern comprise an organic or inorganic material such as glass which remains hard at temperatures greater than 60° C. Any bubbles are again removed preferably via a vacuum. This mixture is then permitted to cure at approximately 60° C. for a minimum of 4 hours. The resulting stamp is released from the master pattern upon curing.

### Methods for Spatially Direct Cellular Growth with the Present Articles.

The methods of the invention can be used to prepare articles that are useful for many purposes including modulating cell-substrate interactions, spatially directing cell growth, tissue regeneration, combinatorial screening strategies, and multiple analytical biosensors. The present methods allow the growth of the cell to be modulated through adhesion and/or growth stimulation along the specific pattern of biologically active molecules on the surface of the article. Patterned polymeric devices prepared in accordance with the methods of this invention are particularly useful in tissue engineering applications where the present micropatterned substrates act as templates to guide and regulate cell growth after promoting cellular adhesion during tissue engineering applications.

The present invention therefore provides methods for spatially modulating the growth of a cell which include contacting a cell with an article prepared by a method of the invention for a time and under conditions sufficient to adhere the cell to the biologically active molecules on the article and to grow the cell along the micron-sized pattern of biologically active molecules on the substrate of the article. This method is suitable for use on any mammal, preferably a human. Any cell type known to one of skill in the art may be used so long as a biologically active molecule can be found which will interact or bind to that cell type. Examples of cell types which can be used include nerve cells, epithelial cells, mesenchymal stem cells, fibroblast cells, and other cell types. In some instances, stem cells are preferred. In a specific embodiment the cell is a nerve cell.

Molecular interactions between neurons and certain biologically active molecules encourage neurite extension. Articles prepared according to the methods of the invention can be beneficial in promoting such neurite extension. Such an article can be patterned as a hollow tube of polymer with biologically active molecules that promote neurite extension adsorbed inside or outside the tube. Such tissue engineering can be initiated outside the body by, for example, removing cells from a patient and seeding those cells onto the article in an appropriate culture medium. When the cells have grown, divided and/or differentiated to form a tissue in culture, the new tissue may be implanted into the body. The article may be implanted at any stage in the growth of the tissue, depending on clinical need and one of skill in the art can readily determine when implantation is appropriate. A biodegradable substrate in the article can be removed by hydrolysis and dissolution in culture before the engineered tissue is implanted into the patient if the function of the substrate is complete. Thus, the substrate can be designed to be completely degraded during in vitro culture or it can be designed to provide support to the bioengineered tissue (for example, a nerve) for a substantially longer predetermined period after surgical implantation.

Two examples of tissue engineering applications in which articles prepared by the invention can be used are directed nerve regeneration and new blood vessel formation (vasculogenesis). For nerve regeneration applications, patterns composed of the biologically active molecules which include peptide sequence Ile-Lys-Val-Ala-Val (SEQ ID NO:2) may be used to encourage nerve cell growth to follow predetermined pathways, i.e. between two severed points of a nerve or towards a de-nerved tissue. For vasculogenesis applications, endothelial cells can be encouraged to grow along patterns of biologically active molecules which include the Arg-Gly-Asp peptide sequence.

A method of forming an article for regenerating tissue according to the present invention may be carried out substantially as described below. First, a biocompatible substrate is chosen which is optionally biodegradable over a desired time period. Second, a type of biologically active molecules or a mixture of biologically active molecules is selected which will provide the desired functions, for example, cell adhesion and/or cell growth. Third, a spatially controlled pattern of the selected biologically active molecules is placed on an activated surface of the substrate. In order to regenerate a tissue, the article can be surgically implanted at the appropriate site or cultured in vitro with surgically removed cells and then surgically implanted, as described above.

An advantage of the present articles is that the non-patterned surface of the substrate can be hydrophobic. This property decreases the ability of cells and other biomolecules to adhere to the non-covered regions of the substrate. Hence, cells adhere more specifically to the regions of the polymeric coated by biologically active molecules and the article is therefore better able to direct cell growth. Additionally, cells may be grown in vitro under common laboratory conditions or in vivo upon implantation of the article into a living creature.

Articles comprising the patterned surfaces prepared via the method of the present invention therefore have various uses. For example, directed growth of neurons is necessary for the repair of peripheral nerve damage. When injury occurs to the nerve, regrowth of the affected axons must be directed along their original path for function to resume. In the body, both physical and chemical cues direct regrowth. Articles prepared in accordance with a method of the present invention can mimic some of these cues thereby encouraging nerve cell alignment and regrowth.

The following non-limiting Examples are provided to further illustrate the invention.

### EXAMPLES

### Example 1

### Master Preparation

Masters used to cast stamps were prepared by methods known in the art. (Moread, W. M., Semiconductor Lithography: Principles and Materials, Plenum, N.Y., 1988; Brambley et al., Adv. Mater. Opt. Electron., 4:55 (1994); Handbook of Microlithography, micromachining, and Microfabrication, Vol. 1 (Ed: P. Rai-Choudbury), SPEI Optical Engineering Press, Bellingham, Wash. (1997)). Briefly, a master may be fabricated on polished silicon wafers using AS P4620 photoresist (Clariant, Inc.) which are spin coated to a thickness of about 5 mm and processed by contact photolithography.

### Example 2

### Polymer Preparation

PMMA (M_{w} 120,000) was obtained from Aldrich Chemical Co. in pellet form and used as received. PMMA (100 mg) was compressed into thin films between highly polished steel plates at 10,000 pounds for 95 seconds using a laboratory press (Carver, Wabash, Ind.) heated to 150° C. The press was then cooled to 90° C., after which the plates were immediately removed and further cooled. The polymer film, with a thickness of 150 to 200 mm, was cut into 2-cm squares.

### Example 3

### Polydimethylsiloxane (PDMS) Stamp Preparation

The master was placed in a petri dish. In a separate container, PDMS monomer (Sylgard 184, Dow Coming, Midland, Mich.) was mixed with the curing agent provided with the PDMS monomer at a 10:1 ratio by weight. Bubbles arising from the mixing process were removed in a vacuum oven (Sheldon MFG, Aloha, Oreg.) at 28″ Hg and at room temperature. This mixture was then poured over the master; any arising bubbles are removed via vacuum at 28″ Hg; and the mixture and master were baked in an oven at 60° C. for a minimum of 4 hours. The resulting PDMS stamp ws released from the master by cutting the PDMS with a sharp blade and peeling it from the master.

### Example 4

### Preparation of Substrates Having Specific Ink Patterns

The following Example illustrates the use of the method of the invention to immobilize aqueous-based biomolecular inks into microscale patterns on organic, biocompatible substrates. The method uses a patterned PDMS stamp to selectively expose or protect underlying substrate regions from the chemical and physical effects of oxygen plasma exposure, thereby forming distinct microscale domains with relatively different hydrophilicities on the substrate. These chemically different areas exhibit varying affinities for a given biomolecule, thereby allowing microscale pattern formation based on the preferential adsorption of ink molecules onto either the plasma-exposed or plasma-protected regions of a particular substrate. The inks evaluated include fluorescein isothiocyanate (FITC)-conjugated goat anti-rabbit immunogobulin G, FITC-conjugated poly-L-lysine, and Texas Red-conjugated bovine serum albumin (BSA). The micropatterned substrates include non-biodegradable polymers such as polyethylene (PE), polystyrene (PS), poly(methyl methacrylate) (PMMA), polydimethylsiloxane (PDMS), and a biodegradable polymer, poly(hydroxybutyrate/hydroxyvalerate) (PHBV). The results demonstrate the stability, under physiologically relevant conditions, of BSA patterns formed by the method of the invention. Additionally, substrates exposed to oxygen plasma while in contact with a patterned PDMS stamp were analyzed by contact angle measurements and scanning electron microscopy to monitor the plasma induced chemical and physical modifications, respectively, occurring on the surfaces.

### Materials and Methods

Substrates. Sheets of polyethylene (PE), polystyrene (PS), poly(methyl methacrylate) (PMMA), and poly(hydroxybutyrate/hydroxyvalerate) (PHBV) were obtained from Goodfellow (Huntingdon, England) and cut into squares (1 cm²) using a razor blade. Polydimethylsiloxane (PDMS) substrates were made using Sylgard 184 silicone elastomer kit (Dow Coming, Midland, Mich.). Elastomer base and curing agent were combined in a 10:1 ratio (w:w), after which prepolymer was placed under vacuum to remove entrapped air. Thin layers of uncured elastomer (˜5 mm) were poured into petri dishes (150 mm×10 mm) and allowed to cure for 24 h under vacuum (−0.98 bar, room temperature). Once cured, PDMS sheets were cut with a razor blade into squares (1 cm²). All substrates were rinsed with HPLC grade ethanol (Sigma-Aldrich, St. Louis, Mo.) prior to patterning.

Biomolecular Inks. Fluorescein isothiocyanate (FITC)-conjugated poly-L-lysine (Sigma, St. Louis, Mo.), FITC-conjugated goat anti-rabbit immunoglobulin G (α-rabbit IgG, Sigma), and Texas Red-conjugated bovine serum albumin (BSA, Molecular Probes, Eugene, Oreg.) were diluted with phosphate buffered saline (PBS, pH 7.4, MP Biomedicals, Aurora, Ohio) to concentrations of 1 mg/mL, 34 μg/mL, and 100 μg/mL, respectively.

Patterned PDMS Stamps. PDMS stamps were prepared by pouring Sylgard 184 silicone elastomer kit (10:1 w:w base to crosslinker) over lithographically created masters, previously described in detail. In brief, masters were created by exposing photoresist-coated silicon wafers through a photomask producing a relief pattern on the silicon surface. The relief pattern consists of a series of raised, parallel lanes (20 μm wide) separated by 20 μm spaces, over which the silicone elastomer was poured. Upon curing, the PDMS was peeled off the master, and the patterned regions were cut using a razor blade into stamps (8 mm×8 mm). To ensure that the ends of the channels would be unobstructed during plasma treatment, the stamps were further trimmed on all sides to remove the outer portions of the patterned regions.

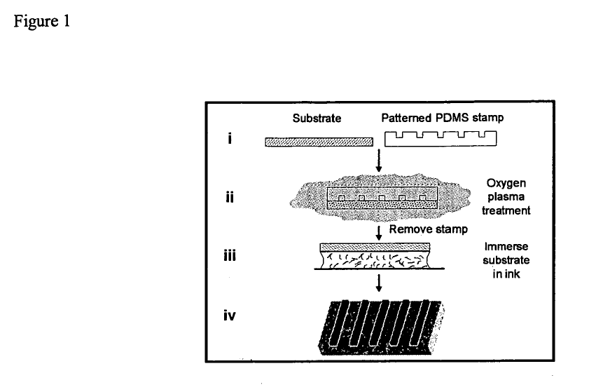

Simple-Pattern Formation. Substrates of PE, PMMA, PS, PDMS, and PHBV were patterned with biomolecular inks (poly-L-lysine, α-rabbit IgG, and BSA) using the procedure outlined in FIG. 1. This method utilizes a striped, patterned PDMS stamp to preferentially expose and protect areas of the underlying substrate to oxygen plasma. The patterned PDMS stamp, consisting of parallel lanes (20 μm) separated by 20 μm spaces, was placed into contact with the substrate, and the entire unit exposed to oxygen plasma (March Plasma, Concord, Calif.) for 300 s at 50 W and 660 mTorr (FIG. 1-ii). Once plasma-treated, the stamp was removed and the substrate immersed, treated side down, in a few drops of the biomolecular ink for no more than 60 s at room temperature (FIG. 1-iii). Typically, substrates were immersed in ink within 60 s after plasma treatment to allow for the biomolecules to adsorb onto either the hydrophobic or hydrophilic regions of the substrate, depending on the specific substrate/ink combination (FIG. 1-iv). Following immersion, the substrates were gently swirled in a beaker of deionized water (˜20 mL) for approximately 10 s to remove non-adsorbed biomolecules, then allowed to air-dry. The biomolecular patterns, consisting of 20 μm stripes separated by 20 μm spaces, were visualized using confocal laser scanning microscopy (CLSM).

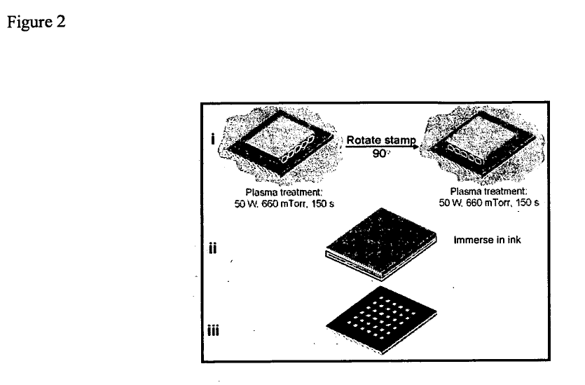

Complex-Pattern Formation. To illustrate the ability of a method of the invention to create more complex micropatterns consisting of distinct regions with differing ink concentrations, PDMS, PMMA, PE, PS, and PHBV substrates were patterned with poly-L-lysine, α-rabbit IgG, or BSA following the method outlined in FIG. 2. The patterned stamp is placed into contact with the substrate and is exposed to oxygen plasma (50 W, 660 mTorr, 150 s). The stamp is removed, rotated 90°, placed back into contact with the substrate, and exposed to plasma a second time (50 W, 660 mTorr, 60 s, FIG. 2-i). Completion of the second step forms four distinct substrate regions: areas exposed to plasma twice, areas first exposed to plasma and then protected in the second step, areas first protected from plasma and then exposed in the second step, and areas protected from the two subsequent plasma treatments. Following the second plasma treatment, the stamp was removed and the substrate immersed, treated side down, in a few drops of the specific ink for no more than 60 s at room temperature (FIG. 2-ii). Following immersion (FIG. 2-iii), the substrates were gently swirled in a beaker of deionized water (˜20 mL) for approximately 10 s to remove non-adsorbed biomolecules and allowed to air-dry. The patterns, ideally consisting of 10×10 μm boxes with different fluorescent intensities, were visualized with CLSM.

To illustrate the effect of different plasma treatments after stamp rotation on pattern formation, PDMS was patterned with poly-L-lysine as follows: the stamp/substrate unit was first exposed to a relatively mild oxygen plasma treatment (50 W, 660 mTorr, 60 s), and then to a more energetic treatment (300 W, 660 mTorr, 120 s) following stamp rotation. The substrate was immersed in ink, rinsed, dried and analyzed as described above.

Dual Ink-Pattern Formation. To demonstrate the ability of μPIP to create micropatterns consisting of distinct, alternating regions of different biomolecules, PMMA was simultaneously patterned with BSA and poly-L-lysine following the method outlined in FIG. 1. However, instead of immersing the plasma-treated substrate in a single ink, the PMMA was immersed in a mixture of BSA and poly-L-lysine (equal parts by volume) for 60 s at room temperature. The substrate was rinsed, dried and analyzed as described above.

Pattern Imaging by CLSM. Simple- and complex-pattern formation was confirmed using a Zeiss LSM 410 CLSM with a computer-controlled laser scanning assembly attached to the microscope. An Omnichrome 3 line Ar/Kr laser operating at 488, 568, and 647 nm was used as the excitation source. The images were processed with Zeiss LSM control software.

Static Contact Angle Measurements. Wettability of substrate areas exposed to or protected from oxygen plasma during treatment was established by contact angle measurements recorded at room temperature using an NRL contact angle goniometer (Rame-Hart). Measurements were taken separately on substrates exposed to oxygen plasma at 50 W and 660 mTorr for 300 s (i.e., “plasma treated exposed”), and substrates that were plasma-treated while in contact with a flat, unpatterned PDMS stamp (i.e., “plasma treated protected”). In each experiment, a drop of deionized, doubly distilled water was placed on the sample, and two contact angle measurements per drop were taken by direct reading. Reported values are an average of at least 6 measurements per sample type.

Evaluation of Substrate and Stamp Topography by Scanning Electron Microscopy (SEM). The surfaces of the substrates following exposure to oxygen plasma (50 W, 660 mTorr, 300 s) while in contact with patterned PDMS stamps, in addition to the PDMS stamps, were imaged with an Amray 1830I scanning electron microscope, using an acceleration potential of 10 kV. Prior to imaging, substrates and patterned stamps were sputter-coated with gold and palladium using a Balzers SCD004 sputter coater (working pressure: 0.05 mbar, working distance: 50 mm, current: 30 mA, time: 120 s).

Evaluation of Pattern Stability. Substrates were micropatterned with BSA following the procedure described in FIG. 1 and imaged with CLSM. Substrates were subsequently immersed in PBS, wrapped in foil, and stored in an incubator at 37° C. for a period of 14 days. The samples were removed from PBS and directly imaged again with CLSM. The samples were stored an additional 14 days under PBS at 37° C., upon which they were imaged with CLSM again, after a total storage time of 28 days.

### Results and Discussion

Simultaneously plasma-treating a substrate, while in physical contact with a patterned PDMS stamp, preferentially increases the hydrophilicity of the exposed substrate regions to produce distinct patterns on the substrate with different relative hydrophilicities determined by the stamp patterns. Once formed, these distinct regions exhibit varying affinities for biomolecules, thereby creating patterns by the preferential attachment of ink molecules to either the plasma-exposed or plasma-protected substrate regions.

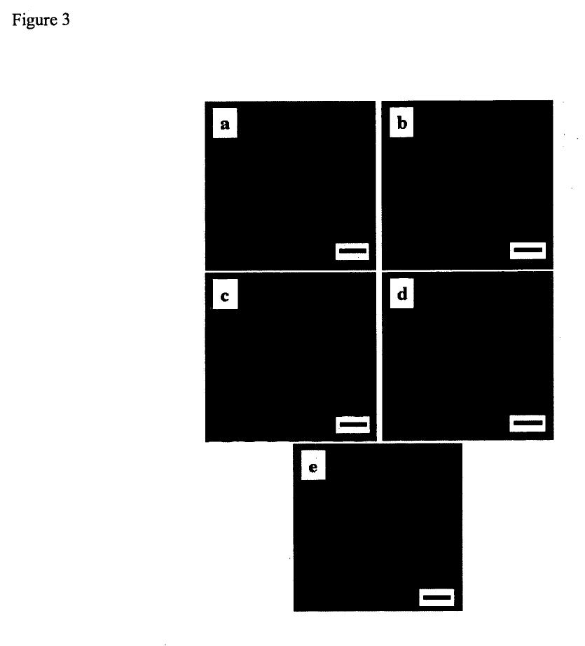

FIG. 3 displays representative fluorescence micrographs of biomolecular inks on polymeric surfaces formed by μPIP, illustrating the applicability of this technique to a varied range of polymers and inks. Generally, the striped patterns were well resolved from the underlying substrate, exhibited uniform ink distribution within the pattern, and had lateral dimensions in good agreement with stamp features. Although the striped patterns shown in FIG. 3 were formed after immersion in ink for 60 s, pattern formation was observed even after 5 s of immersion. However, the quality and uniformity of the resulting patterns were not as consistent at short immersion relative to longer immersion times. Immersion times significantly longer than 60 s (e.g., 60 min), resulted in patterns with poorer resolution due to significant ink adsorption in both plasma-protected and plasma-exposed regions. Thus, immersion time, as well as plasma-treatment parameters (e.g., feed gas, power, duration, chamber pressure), are two key variables that can be optimized for highly resolved patterns for a given ink/substrate combination.

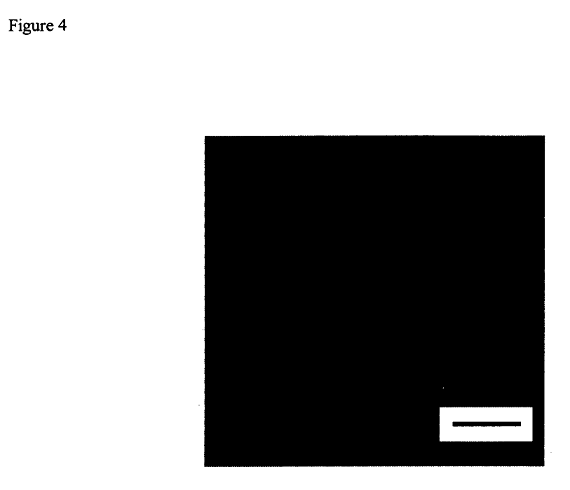

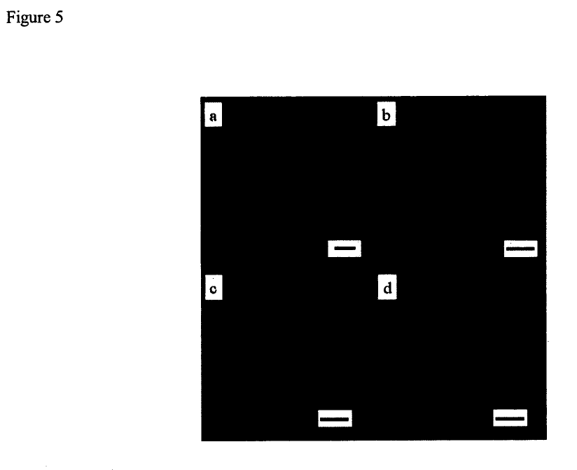

One significant advantage of this technique is that pattern formation is widespread, occurring wherever the stamp is in good contact with the substrate (FIG. 4). Another benefit of this method is the ability to easily produce more complex patterns than simple stripes. FIG. 5 illustrates the effect of dual plasma treatments/stamp orientations on ink adsorption and resulting complex-pattern formation.

Plasma-treating the substrates as outlined in FIG. 2 creates four distinct zones: areas exposed to plasma twice, areas first exposed to plasma and then protected in the second step, areas first protected from plasma and then exposed in the second step, and areas protected from the two subsequent plasma treatments. However, as the same plasma treatment is used after stamp rotation, regions that are exposed to plasma only once are chemically modified to the same extent and should exhibit similar affinities for a given ink compared to each other, but different affinities as compared to regions exposed to plasma twice or protected from plasma twice. As shown in FIGS. 5a-c, substrates with three distinct concentrations of the ink were formed. The relative orientations of these regions change depending on the ink/substrate combination and/or with the plasma-treatment parameters used. For example, the effect of ink/substrate combination can be shown by comparing the fluorescent micrographs of FIG. 5. The substrates in FIGS. 5a-c were treated using the same oxygen plasma treatment (50 W, 660 mTorr, 150 s, rotate 90°, 50 W, 660 mTorr, 150 s), yet the resulting patterns on PMMA (FIG. 5a) appear strikingly different from those on PDMS (FIG. 5b) and PS (FIG. 5c). Rather than patterns consisting of lines with the weakest fluorescence alternating with lines that exhibit intermediate and strongest fluorescence (FIGS. 5b-c), a “plaid” pattern is formed on PMMA (FIG. 5a). FIG. 5d shows the effect of employing two different plasma treatments on pattern formation. The substrate was exposed to a mild treatment (50 W, 660 mTorr, 60 s), and then with a relatively more energetic treatment (300 W, 660 mTorr, 120 s) after stamp rotation. In contrast to the patterns in FIGS. 5a-c, stripes with uniform fluorescence are created, rather than forming alternating boxes of differing fluorescence. This result indicates that the second, more energetic plasma-treatment modified the exposed regions to the same extent, regardless of the initial modifications introduced by the first treatment.

FIGS. 3-5 illustrate the versatility of this technique and also highlight the many factors that contribute to pattern formation; treatment parameters can be optimized based on the ink/substrate combination, as a given ink cannot be expected to behave the same way on two different substrates. For example, BSA molecules attach to the plasma-exposed regions of PHBV, whereas BSA attaches to the plasma-protected regions of PS; the specific molecular interactions at the BSA/PHBV interface are obviously different than the BSA/PS interface. The specific molecular interactions at the various ink/substrate interfaces are currently under investigation and beyond the scope of this manuscript. Yet, preliminary information on these interactions is obtained from the stability of BSA-patterned substrates upon storage.

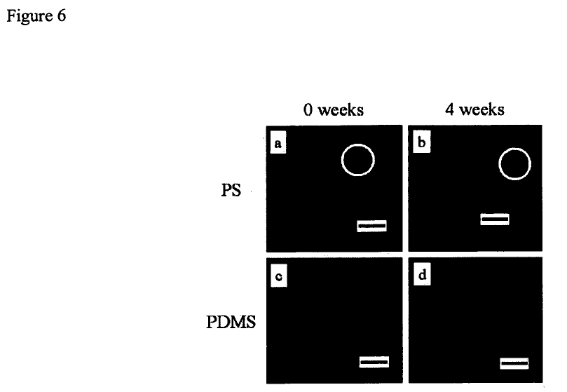

The micrographs in FIG. 6 highlight the stability of BSA on substrates following four weeks of storage in PBS at 37° C. Images for PS (FIGS. 6a-b) were taken from the same area of the substrate, whereas images for PDMS (FIGS. 6c-d) were taken from different areas of the same substrate. The BSA patterns on PS remained intact and exhibited no changes in stripe width, indicating good stability at physiologically relevant conditions for at least four weeks (FIG. 6b). In fact, pattern resolution was actually improved upon storage: the faint fluorescence between the BSA stripes shown in FIG. 6a disappeared, indicating that longer incubation removed weakly adsorbed ink molecules on the “unpatterned” regions. The encircled area of FIG. 6b is not a result of pattern loss on storage, as this region was originally present (encircled area, FIG. 6a), and is likely a result of an anomaly on the PDMS stamp surface. BSA patterns on PE, PMMA, and PHBV (not shown) were equally stable after four weeks; these substrates displayed well resolved, distinct striped patterns that exhibited improved pattern resolution upon long term storage. In contrast, BSA patterns on PDMS, though present and intact, exhibited significant pattern-widening and diffusion after four weeks. This loss of pattern resolution was not observed after two weeks of storage, indicating that these patterns are stable for a period of somewhere between two and four weeks. As stated previously, the difference in pattern stability is likely due to differences in molecular interactions at the ink/substrate interface.

TABLE 1
Water Contact Angles of Polymer Substrates After Oxygen Plasma
Treatment (50 W, 660 mTorr, 300 s): Protected vs. Exposed

| | Plasma-treated | Plasma-treated | |
|---|---|---|---|
| | Protected | Exposed | Differential |
| PDMS | 111.4 ± 3.7° | 35.4 ± 5.4° | 76.0° |
| PMMA | 83.4 ± 9.8° | 38.8 ± 4.4° | 44.6° |
| PS | 91.0 ± 4.2° | 21.0 ± 2.1° | 70.0° |
| PE | 81.1 ± 9.8° | 45.0 ± 2.6° | 36.1° |
| PHBV | 81.5 ± 2.8° | 31.4 ± 2.8° | 50.1° |

Comparison of “plasma-treated protected” and “plasma-treated exposed” contact angles in Table 1 shows that exposure to oxygen plasma significantly increased the hydrophilicity of each polymer substrate, with PDMS exhibiting the largest change in contact angle (˜76°) and PE showing the smallest decrease (˜36°). It is well established that oxygen plasma treatments increase the hydrophilicity of a polymer surface by chemically incorporating polar, oxygen-containing functionalities. Clearly, plasma treatment of polymer substrates in the fashion outlined in FIG. 1 creates distinct regions with significantly different hydrophilicities that enables distinct patterns on various polymer substrates (as shown in FIGS. 3-5).

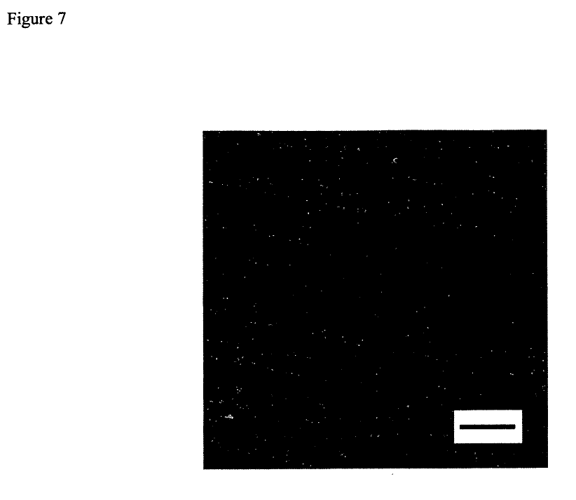

To further verify that distinct regions with significantly different hydrophilicities are formed on polymer substrates by the method in FIG. 1, dual ink patterns were formed on PMMA by immersion of the substrate in a mixture of poly-L-lysine and BSA (equal parts by volume). As shown in FIG. 7, the biomolecules spontaneously segregated and preferentially adsorbed to either the plasma-exposed (i.e., relatively hydrophilic) or plasma-protected (i.e., relatively hydrophobic) regions of the substrate.

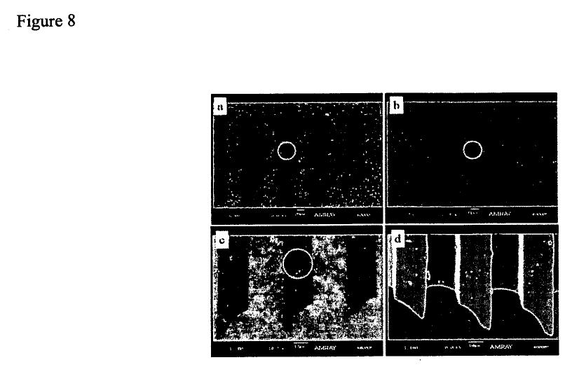

Exposure to oxygen plasma physically modifies the polymer surfaces, in addition to increasing hydrophilicity. SEM micrographs (FIGS. 8a-c) show two distinct regions when plasma treating polymer substrates in contact with a patterned PDMS stamp. Areas exposed to plasma, which appear lightened in FIGS. 8a-c, were rougher compared to adjacent, plasma-protected regions, which are the darker regions (encircled areas in FIGS. 8a-c). The resulting topographies of PMMA, PS, PE, and PHBV are consistent with those of FIGS. 8a and 8c, which show clear delineation of the differently exposed surfaces. In contrast, the delineation was more subtle in the case of PDMS (FIG. 8b). Instead of increased roughening relative to the plasma-protected regions, the plasma-exposed regions of PDMS exhibited a relatively smooth surface interspersed with numerous cracks. This cracking has been well documented and is consistent with prior studies of PDMS surfaces exposed to plasma. The ability to physically modify polymer surface topography by oxygen plasma is well documented, and has been attributed to the chemical and physical etching by radical reactions and ion bombardment, respectively.

FIGS. 8c and 8d show the topography of the bottom-left region of a PHBV substrate and the corresponding bottom-right region of the patterned PDMS stamp in contact with the substrate during plasma treatment, respectively. The micrographs, which are mirror images of each other, illustrate the excellent agreement between the spatial dimensions/features of the PDMS stamp pattern and the resulting pattern on the substrate. These results support the contention that the underlying substrate surface is effectively shielded from the oxidative effects of plasma in the regions that make sufficient contact with the PDMS stamp.

1. A method comprising: providing a surface including a plurality of areas having different surface energies; and contacting the surface with an ink that preferentially adheres to one area.

2. The method of claim 1 wherein the surface comprises a glass.

3. The method of claim 1 wherein the surface comprises a polymer.

4. The method of claim 3 wherein the polymer comprises polycarbonate, poly(hydroxy acid), polyanhydride, polyorthoester, polyphosphazene, polyphosphate, polyester, or a mixture thereof.

5. The method of claim 3 wherein the polymer comprises polyacrylate, polymethylacrylate, polymethacrylate, polyethylene, polystyrene, poly(methyl methacrylate), polydimethylsiloxane, polystyrene, or Nylon 6,6.

6. The method of claim 3 wherein the polymer comprises polycarbonate, poly(hydroxy acid), polyanhydride, polyorthoester, polyphosphazene, polyphosphate, polyester, polyacrylate, polymethylacrylate, polymethacrylate, polyethylene, polystyrene, poly(methyl methacrylate), polydimethylsiloxane, polystyrene, or Nylon 6,6, or a mixture thereof.

7. The method of claim 1 wherein the surface has two areas having different surface energies.

8. The method of claim 1 wherein the ink comprises a hormone, an extracellular matrix molecule, a cell adhesion molecule, a natural polymer, an enzyme, a peptide, an antibody, an antigen, a polynucleotide, a growth factor, a synthetic polymer, polylysine, or a drug.

9. The method of claim 1 wherein the ink is goat anti-rabbit IgG, poly-L-lysine, PHB/HV, or laminin.

10. The method of claim 1 wherein the surface including a plurality of areas having different surface energies is prepared by providing a surface that has a protected portion and an unprotected portion; and treating the surface under conditions that alter the surface energy of the unprotected portion to provide a surface including a plurality of areas having different surface areas.

11. The method of claim 1 wherein the surface including a plurality of areas having different surface energies is prepared by: contacting a stamp having a pre-selected pattern with a surface to provide a surface that has a portion that is protected by the stamp and an unprotected portion; and treating the surface under conditions that alter the surface energy of the unprotected portion to provide a surface including two areas having different surface energies.

12. The method of claim 11 wherein the stamp is removed from contact with the surface prior to contact with the ink.

13. The method of claim 11 wherein the stamp is removed from contact with the surface after contact with the ink.

14. The method of claim 1 further comprising, washing the surface to remove non-adhered ink from the surface.

15. The method of claim 1 wherein the ink adheres in a pre-selected pattern on the surface.

16. The method of claim 15 wherein the pattern is micron-sized.

17. The method of claim 15 wherein the substrate is biodegradable.

18. The method of claim 1 wherein the ink comprises biologically active molecules that inhibit cell adhesion, growth or differentiation.

19. The method of claim 1 wherein the ink comprises 2-10 different types of biologically active molecules.

20. The method of claim 15 wherein the pre-selected pattern is a line.

21. The method of claim 20 wherein the line is from about 5 micron to about 50 microns in length.

22. The method of claim 20 wherein the line is from about 1 to about 50 nm in width.

23. A method comprising: contacting a stamp having a pre-selected pattern with a surface on a hydrophobic polymeric substrate to provide a surface that has a portion that is protected by the stamp and an unprotected portion; treating the surface under conditions that alter the polarity of the unprotected portion to provide a surface including two areas having different polarities; and contacting the surface including two areas having different polarities with an ink that comprises a biologically active molecule that adheres preferentially to one of the two areas to provide a hydrophobic polymeric substrate having a pattern of biologically active molecules thereon.

24. The method of claim 11 wherein said conditions that alter the polarity of the unprotected portion comprise exposure to a plasma.

25. The method of claim 24 wherein the plasma comprises argon, nitrogen, or oxygen plasma.

26. An article made by the method of claim 1.

27. The method of claim 3, wherein the polymer is a biodegradable polymer.

28. The method of claim 23 further comprising removing the stamp from contact with the surface after the contact with the ink.

29. The method of claim 23 further comprising removing the stamp from contact with the surface prior to the contact with the ink.

30. The method of claim 23 further comprising washing non-adhered ink from the surface.
